(19) United States
(12) Patent Application Publication
(10) Pub. No.: US 2022/0340871 A1
(43) Pub. Date: Oct. 27, 2022

(54) METHOD FOR OBTAINING OR MAINTAINING ABCG2-POSITIVE CORNEAL LIMBAL STEM CELLS

(71) Applicant: STEMSIGHT OY, Lempäälä (FI)

(72) Inventors: Meri VATTULAINEN, Tampereen yliopisto (FI); Keijo VIIRI, Tampereen yliopisto (FI); Tanja ILMARINEN, Tampereen yliopisto (FI); Heli SKOTTMAN, Tampereen yliopisto (FI)

(21) Appl. No.: 17/600,457

(22) PCT Filed: Mar. 31, 2020
(86) PCT No.: PCT/FI2020/050210

§ 371 Date: Sep. 30, 2021

(30) Foreign Application Priority Data
Apr. 1, 2019 (FI) 20195258

(51) Int. Cl. C12N 5/079

(52) U.S. Cl. CPC C12N 5/0621; C12N 2501/11; C12N 2501/415; C12N 2501/998

(57) ABSTRACT

Disclosed is a method of producing ABCG2-positive corneal limbal stem cells through inducing pluripotent stem cells first into eye precursor cells and then differentiating the eye precursor cells into ABCG2-positive corneal limbal stem cells. Also disclosed is a method of maintaining ABCG2-positive phenotype of corneal limbal stem cells, such as primary corneal limbal stem cells.

### BACKGROUND OF THE INVENTION

### Field of the Invention

The present description relates to differentiation of pluripotent stem cells into eye precursor cells and further into ABCG2-positive limbal stem cells. If desired, the cells may be differentiated further towards highly proliferative ΔNp63α-positive limbal progenitors and finally into corneal epithelial cells. The invention also relates to a method of maintaining ABCG2-positive phenotype of corneal limbal stem cells, such as primary corneal limbal stem cells or corneal limbal stem cells obtained by the differentiation method.

### Description of the Related Art

Human cornea is a multilayered transparent connective tissue acting as a protective barrier on the front surface of the eye and allowing us a clear vision. The outermost corneal epithelium has a rapid renewal cycle that appears via constant cell loss and replacement on the ocular surface. This turnover of epithelial layers is enabled by limbal stem cells (LSCs) that reside in specific anatomical niche structures in the Palisades of Vogt and supply the epithelium with new cells. LSC dysfunction or loss results in a clinical condition named limbal stem cell deficiency (LSCD), which can ultimately lead to corneal blindness. Unfortunately, its efficient treatment with LSC transplants is hampered due to worldwide shortage of suitable donor tissue. Therefore, several protocols have been developed with the aim to provide an alternative, inexhaustible cell source by differentiating LSCs from human pluripotent stem cells (hPSCs), including both human embryonic and induced pluripotent stem cells (hESCs and hiPSCs, respectively). Importantly, even though many laboratories still utilize animal-derived components that impede the clinical translation of these methods, also a few clinically relevant defined and feeder cell-free techniques have been introduced (Hongisto et al. 2017 Stem Cells Res Ther; Mikhailova et al. 2014 Stem Cell Rep).

In addition to highly varying practices, another critical challenge existing in the field is the identification of the clinically relevant LSC population. While several proteins have been associated with putative LSCs in the limbal basal epithelium, no specific marker has been found for definite discrimination of these cells. Expression of p63α has been linked to success in clinical transplantations, and is currently regarded as the most promising marker for clinical relevancy (Rama et al. 2010, N Engl J Med, 363:147-55).

WO 2018/037161 and Hongisto et al. 2017 Stem Cells Res Ther discloses a method of differentiating human pluripotent stem cells obtained from a feeder-free culture first into eye precursor cells and then into p63-positive corneal limbal epithelial precursor cells. The method comprises an induction phase wherein the pluripotent stem cells are first cultured in the presence of a TGF-beta inhibitor and fibroblast growth factor (FGF) and then in the presence of bone morphogenetic protein 4 (BMP-4). The eye precursor cells so obtained are then differentiated into p63-positive corneal epithelial precursor cells using a cell culture medium comprising one or more supplements selected from the group consisting of epidermal growth factor (EGF), hydrocortisone, insulin, isoproterenol, and tri-iodo-thyronine. Optionally, the p63-positive corneal epithelial precursor cells are then allowed to mature into mature corneal epithelial cells or into corneal stratified epithelium.

In the cornea, expression of ATP-binding cassette sub-family G member 2 (ABCG2) is exclusively located in the limbal basal layers and has been associated with an immature and quiescent LSC subpopulation (SP) phenotype, marked by the ability to efflux Hoechst 33342 dye. In culture, these cells activate and express a greater growth potential compared to ABCG2-negative cells. (De Paiva et al. 2007, Stem Cells).

Further understanding of the differentiation hierarchy and functional roles of a wide variety of proteins expressed in the limbal basal epithelium would significantly increase both the efficacy and safety of future LSC transplantations. The identification of LSCs currently relies on the co-occurrence of several positive markers, combined with the absence of cytokeratins (CK) 3 and 12 that are the markers of terminally differentiated corneal epithelia (CE) (Schlötzer-Schrehardt & Kruse 2005 Exp Eye Res). Apart from a few studies, the mutual relations of these markers and their exact positions in functional hierarchy have remained largely unknown.

Nevertheless, ABCG2 has been acknowledged as a promising marker for a potent stem cell phenotype in vivo and could therefore be used for identification of clinically relevant LSCs. Thus, there is a need for methods for obtaining ABCG2-positive LSCs, which may be used for treating and studying corneal epithelial conditions, diseases, and pathologies. In addition, such cells may also be used in toxicological studies and drug development, especially in cases where the stem cells are obtained from cell cultures lacking feeder cells. Further, avoidance of xeno-derived or undefined components is also an important aim that improves the safety of potential clinical translation of these methods.

### SUMMARY OF THE INVENTION

An object of the present invention is to provide ABCG2-positive corneal limbal stem cells and methods of producing and maintaining the same so as to overcome the problems associated with easily lost expression of ABCG2 in cell culture conditions. The object of the invention is achieved by the method which are characterized by what is stated in the independent claims. The preferred embodiments of the invention are disclosed in the dependent claims.

Accordingly, the present invention provides a method of maintaining ABCG2-positive phenotype of corneal limbal stem cells, wherein the method comprises culturing ABCG2-positive corneal limbal stem cells in a culture medium comprising EGF and at least one Wnt activator. In some embodiments, the Wnt activator is selected from the group consisting of GSK3 inhibitors, preferably CHIR99021, and proteins of R-spondin family, preferably R-spondin-1 or its supplement RS-246204. In some further embodiments, the culture medium may also comprise Noggin or its supplement, such as LDN-193189.

In addition, the present invention also provides a method of producing ABCG2-positive corneal limbal stem cells, the method comprising:

A method of producing ABCG2-positive corneal limbal stem cells, the method comprising: a) providing pluripotent stem cells; b) culturing said cells in a cell culture medium comprising a TGF-beta inhibitor and a fibroblast growth factor (FGF), preferably basic FGF; c) withdrawing the TGF-beta inhibitor and the FGF, and culturing the cells obtained in step b) in a cell culture medium comprising bone morphogenetic protein 4 (BMP-4) thereby producing eye precursor cells; d) culturing said eye precursor cells in a corneal differentiation medium thereby producing ABCG2-positive corneal limbal stem cells; and e) culturing said ABCG2-positive corneal limbal stem cells in a maintenance cell culture medium comprising EGF and at least one Wnt activator thereby maintaining the ABCG2-positive limbal stem cell phenotype obtained via steps a-d). In some embodiments, the Wnt activator is selected from the group consisting of GSK3 inhibitors, preferably CHIR99021, and proteins of R-spondin family, preferably R-sponding-1 or its supplement RS-246204. In some further embodiments, the culture medium may also comprise noggin or its supplement, such as LDN-193189.

Further aspects, specific embodiments, object, details, and advantages of the invention are set forth in the following drawings, detailed description, and examples.

### BRIEF DESCRIPTION OF THE DRAWINGS

In the following the invention will be described in greater detail by means of preferred embodiments with reference to the attached drawings, in which

FIG. 1. Schematic drawing of the present methods. FIG. 1A illustrates one embodiment of a method of producing ABCG2-positive corneal limbal stem cells from pluripotent stem cells. FIG. 1B illustrates one embodiment of a method of maintaining ABCG2-positive phenotype of primary corneal limbal stem cells.

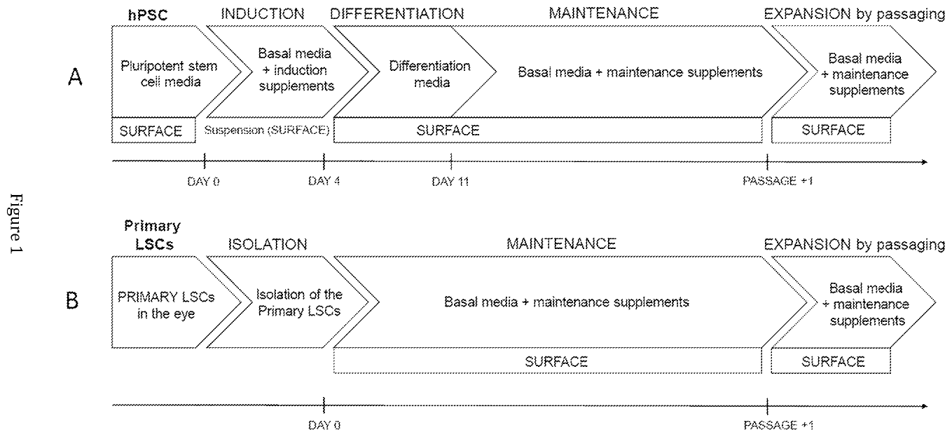

FIG. 2. Characterization of the putative LSC marker expression during hPSC-LSC differentiation. (FIG. 2A) Representative morphology and protein expression of the hPSC-LSC culture in selected time points, as demonstrated by IF. Scale bars 100 μm apply for all images in the same column. Cell nuclei were counterstained with DAPI, shown in the upper-right corner of each panel. (FIG. 2B) Marker expression differences in day 10 and day 24 hPSC-LSC populations. Five images per sample and a minimum of 1400 cells per time point were analyzed for each marker from cytospin samples. Data are presented as mean+SD, n=2-3 individual cell differentiations. (FIG. 2C) Representative IF image of ΔNp63 and p63α double-staining in day 24 cytospin sample, confirming the ΔNp63α-positive phenotype. Scale bar 100 μm applies for both C and D. (FIG. 2D) Representative IF image of ABCG2 and p63α double-staining in day 10 cytospin sample, demonstrating the colocalization pattern. (FIG. 2E) p63α and ABCG2 expression and their colocalization in day 10 and day 24 hPSC-LSCs, analyzed from cytospin samples. Five images per sample and a minimum of 3000 cells per time point were analyzed from double-stained cytospin samples. Data are presented as mean+SD, n=3 individual cell differentiations. (FIG. 2F) The level of ABCG2 protein expression in UD-hPSCs, day 10 and day 24 hPSC-LSCs, analyzed with FACS from 10 000 recorded events for each sample. Data are presented as mean+SD, n≥3 individual cell differentiations, statistics calculated with Mann-Whitney U-test. (FIG. 2G) The relative ABCG2 mRNA expression levels in UD-hPSCs, day 10 and day 24 hPSC-LESCs analyzed with qRT-PCR using three technical replicates for each sample. Data are presented as mean+SD, n=2 individual cell differentiations. All representative data is presented with hESC line Regea08/017.

FIG. 3. Effect of culture condition on hPSC-LSC morphology and p63α/ABCG2 expression. Representative cell morphology as well as p63α and ABCG2 protein expression after continued culture in corneal differentiation (CnT-30) condition versus ABCG2-positive LSC maintenance (Cnt-07+ENRC) condition at day 21 (of total culture time) (FIG. 3A). P63α and ABCG2 protein expression pattern was preserved after passaging the cells in Cnt-07+ENRC condition (FIG. 3B). Relative ABCG2 mRNA expression of hPSC-LSCs after continued culture in corneal differentiation (CnT-30) condition versus ABCG2-positive LSC maintenance (Cnt-07 +ENRC) condition at day 21 (of total culture time) (FIG. 3C). All representative data is presented with hESC line Regea08/017. Scale bars in all images 100 μm.

FIG. 4. Marker expression and proliferative capacity of ABCG2-positive hPSC-LSCs during passaging. (FIG. 4A) Human PSC-LSC colonies retain their morphology and p63α/ABCG2 expression pattern over passaging in CnT-07+ENRC condition. Black scale bars 200 μm and white scale bars 100 μm. 20,20 population doublings in the course of five passages were achieved without definite signs of culture exhaustion as demonstrated by steady population doubling times (FIGS. 4B-C, black dots). Cryopreservation of the cells at passage 3 did not seemingly affect the proliferative capacity of the hPSC-LSCs (FIGS. 4B-C, white boxes). All representative data is presented with hESC line Regea11/013.

### DESCRIPTION OF THE PREFERRED EMBODIMENTS

The present invention provides a method of producing ABCG2-positive corneal limbal stem cells through inducing pluripotent stem cells first into eye precursor cells (in an induction phase) and then differentiating the eye precursor cells into ABCG2-positive corneal limbal stem cells (in a differentiation phase) and then maintaining the ABCG2 expression (in a maintenance phase). Also provided is a method of maintaining ABCG2-positive phenotype of corneal limbal stem cells, including primary corneal limbal stem cells and pluripotent stem cell-derived corneal limbal stem cells. Furthermore, the invention relates to the therapeutic use of the ABCG2-positive cells obtained or maintained in accordance with the invention.

### Cells

As used herein, the term “corneal limbal stem cells” refers to adult stem cells located in the basal epithelial layer of the corneal limbus and responsible for repopulating the corneal epithelium. The term is interchangeable with the terms “limbal stem cells (LSC)”, “limbal epithelial stem cells (LESC)”, “corneal epithelial stem cells” and “corneal epithelial precursor cells”. As used herein, the terms “precursor” and “progenitor” may be used interchangeably unless otherwise indicated.

As used herein, the term “primary corneal limbal stem cells” refers to corneal limbal stem cells taken directly from living tissue. Means and method for obtaining primary corneal limbal stem cells are readily available in the art.

As used herein, the term “pluripotent stem cell-derived corneal limbal stem cells” refers to corneal limbal stem cells derived from pluripotent stem cells. In some embodiments, the present invention provides a method of producing ABCG2-positive corneal limbal stem cells through inducing pluripotent stem cells first into eye precursor cells (induction phase) and then differentiating the eye precursor cells into ABCG2 -positive corneal limbal stem cells (differentiation phase) that can, if desired, be allowed to further differentiate towards highly proliferative ΔNp63α-positive limbal epithelial progenitors and finally mature corneal epithelial cells.

As used herein, the term “pluripotent stem cell” refers to any stem cell having the potential to differentiate into all cell types of a human or animal body, not including extra-embryonic tissues. These stem cells include both embryonic stem cells (ESCs) and induced pluripotent cells (iPSCs). Hence, the cells suitable for use in the present invention include stem cells selected from iPSCs and ESCs. Human pluripotent stem cells (hPSCs) are preferred and they include human iPSCs (hiPSCs) and human ESCs (hESCs).

ESCs, especially hESCs, are of great therapeutic interest because they are capable of indefinite proliferation in culture and are thus capable of supplying cells and tissues for replacement of failing or defective human tissue. However, producing eye precursor cells from human embryonic stem cells may meet ethical challenges. According to an embodiment of the present invention, human embryonic stem cells may be used with the proviso that the method itself or any related acts do not involve destruction of human embryos.

Induced pluripotent stem cells, commonly abbreviated as iPS cells or iPSCs are a type of pluripotent stem cell artificially derived from a non-pluripotent cell, typically an adult somatic cell, by inducing a forced expression of specific genes by means and methods well known in the art. An advantage of using iPS cells is that no embryonic cells have to be used at all, so ethical concerns can be avoided. A further advantage is that production of patient-specific cells without immunorejection problems is enabled by employing iPSC technology. Therefore, according to an embodiment of the present invention, use of iPS cells is preferred. For clinical use, hiPS cells are preferred.

Induced pluripotent stem cells are similar to natural pluripotent stem cells, such as embryonic stem cells, in many aspects. Exemplary aspects include the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed. Induced pluripotent cells are typically made from adult skin cells, blood cells, stomach or liver, although other alternatives may be possible. Those skilled in the art are familiar with the potential of iPS cells for research and therapeutic purposes.

Pluripotent stem cells are difficult to maintain in cell cultures because they tend to follow their natural cell fate and differentiate spontaneously. To prevent unwanted differentiation, pluripotent stem cells are typically cultured on feeder cells. Animal-derived feeder cells, such as mouse embryonic fibroblasts (MEFs), are widely used as feeder cells even for human pluripotent cells. To replace animal-derived materials, human-derived feeder cells, such as human foreskin feeder cells, have also been employed for culturing human pluripotent stem cells. Accordingly, in some embodiments, pluripotent stem cells cultured on feeder cells, preferably on human feeder cells, such as human foreskin feeder cells, may be employed in the present method of producing ABCG2 -positive corneal limbal stem cells.

To overcome drawbacks associated with the use of feeder cells, new feeder-free cell culturing methods have been developed. Accordingly, the term “feeder-free culture” or any linguistic variation thereof, refers to a culture of pluripotent stem cells in the absence of any feeder cells.

Use of feeder cells can be omitted, for example, by replacing them with an appropriate substrate coating as is well known in the art. Suitable coating materials include, but are not limited to, human or animal, natural extracted or recombinant extracellular matrix (ECM) proteins such as laminins, collagens, vitronectin, fibronectin, nidogens, proteoglycans, and E-cadherin, as well as isoforms, fragments, and peptide sequences thereof. Non-limiting examples of said

ECM protein isoforms include different isoforms of laminin, such as Laminin-511, -521, -322, -411 etc. Non-limiting examples of said fragments include E8 fragments of human laminin isoforms, whereas one non-limiting example of said peptide sequences is an Arg-Gly-Asp (RGD) sequence of vitronectin. Said ECM proteins, isoforms, fragments, or peptide sequences may also be fused to other proteins, N-cadherin domain fused to an IgG-Fc domain being one non-limiting example of such fusion proteins. Alternative or additional suitable coating materials include, but are not limited to, ECM or basement membrane extracts from different tissue or cell types of human or animal origin, such as mouse embryonic fibroblast, human fibroblasts, mesenchymal stem cells, and tumours. Further suitable coating materials include natural or synthetic biomaterials and hybrids thereof, either alone or as functionalised with ECM proteins or sequences thereof or other chemical or physical surface modifications. Non-limiting examples of such biomaterials include PMEDSAH (poly[2 imethacryloyloxy)ethyl-dimethyl-(3-sulfopropyl)ammonium hydroxide) and collagen-grafted Mixed Cellulose Esters membrane (MCE-COL). Non-limiting examples of further suitable coating materials include commercial products such as Corning® Synthemax® surface, Corning® PureCoat™, CELLstart™, Matrigel™, or Geltrex®. Any of the above-mentioned proteins, isoforms, fragments, peptide sequences, fusion proteins, extracts, biomaterials or commercial products may be used either alone or in any appropriate combinations or mixtures to replace feeder cells as is well known in the art. Means and methods for obtaining, selecting, and using suitable coating materials for replacing feeder cells are readily available in the art.

In accordance with the above, pluripotent stem cells cultured on feeder-free conditions may be employed in some embodiments of the present method of producing ABCG2-positive corneal limbal stem cells.

Preferred extracellular matrix proteins include laminins, heterotrimeric glycoproteins that contain a α-chain, a β-chain, and a γ-chain, found in five, four, and three genetic variants, respectively. The laminin molecules are named according to their chain composition. Thus, as used herein, the term “laminin-521” refers to a laminin containing α5, β2, and γ1 chains, whereas the term “laminin-511” refers to a laminin containing α5, β1, and γ1 chains. Preferred laminins include recombinant human laminin-521, recombinant human laminin-511, and fragment E8 of recombinant human laminin-511.

While human pluripotent stem cells grow as colonies on feeder cells, they grow in a feeder-free culture on laminin-521 as a monolayer allowing more effective expansion. Moreover, according to flow cytometric analyses, human pluripotent stem cells cultured in a feeder-free culture system show greater positivity to pluripotency markers as compared with cells cultured on feeder cells.

Indeed, human pluripotent stem cells obtained from a culture containing feeder cells or from a feeder-free culture appear to be different. As demonstrated in the experimental part of WO 2018/037161, corneal differentiation method disclosed in EP 2828380 perform well with human pluripotent stem cells obtained from a culture comprising feeder cells, but it does not perform equally well with pluripotent cells obtained from a feeder-free culture. On the other hand, the method of WO 2018/037161 provides excellent differentiation of human pluripotent stem cells obtained from a feeder-free culture first into eye precursor cells and the into p63-positive corneal epithelial precursor cells (i.e. corneal limbal stem cells). The present invention, in turn, provides excellent maintenance of ABCG2-positive corneal limbal stem cells. Thus, in some embodiments of the present invention, undifferentiated pluripotent stem cells obtained from a feeder-free culture are used for producing ABCG2-positive corneal limbal stem cells. However, the present invention is not limited to differentiation of human pluripotent stem cells obtained from a feeder-free culture, but may also be used for differentiating pluripotent stem cells cultured on feeder cells into ABCG2-positive corneal limbal stem cells.

As used herein, the term “eye precursor cell” refers broadly to any cell lineage of the eye induced from pluripotent stem cells and characterized by down-regulation of the pluripotency marker OCT-4 (also known as POU5F1) and up-regulation of PAX6, a gene indicating differentiation into eye specific cell lineages. Means and methods for quantifying pluripotency markers are readily available in the art.

As used herein, the term “p63-positive corneal limbal stem cells” refers to a population of corneal limbal stem cells that express p63 on their cell surfaces.

As used herein, the term “ABCG2-positive corneal limbal stem cells” refers to a population of corneal limbal stem cells that express ATP-binding cassette sub-family G member 2 (ABCG2) on their cell surfaces.

Cell surface markers, such as ABCG2 and p63, may be quantified by any available technique suitable for this purpose including, but not limited to, immunofluorescence and flow cytometry, such as fluorescence activated cell sorting (FACS).

### Method of Producing ABCG2-Positive Corneal Limbal Stem Cells

WO 2018/037161 discloses a method of producing differentiated eye cells by inducing pluripotent stem cells obtained from a feeder-free culture into eye precursor cells first in the presence of a TGF-beta inhibitor and fibroblast growth factor (FGF) and then in the presence of bone morphogenetic protein 4 (BMP-4). The eye precursor cells so obtained are then differentiated into p63-positive corneal epithelial precursor cells (p63-positive corneal limbal stem cells) by withdrawing the TGF-beta inhibitor, FGF and BMP-4, and culturing the eye precursor cells in the presence of one or more supplements selected from the group consisting of epidermal growth factor (EGF), hydrocortisone, insulin, isoproterenol, and tri-iodo-thyronine. Optionally, the p63-positive corneal epithelial precursor cells are then allowed to mature into mature corneal epithelial cells or into corneal stratified epithelium.

Unexpectedly, careful analysis of the expression patterns of several pluripotency markers, limbal stem cell markers as well as corneal epithelial markers at different time points throughout the method of WO 2018/037161 revealed that ABCG2 was only transiently expressed, peaking at day 10-11 and then gradually decreasing to very low levels by day 21 of the differentiation protocol (including the 4-day induction period). Simultaneously, the expression of ΔNp63α isoform of p63 steadily increased. In addition, it was unexpectedly realized that strong ABCG2 expression can be maintained until at least day 35 without passaging and until at least 50 days with passaging subconfluent cultures by supplementing the culture medium with at least epidermal growth factor (EGF) and at least one Wnt activator.

Thus, the present invention provides a method of producing and maintaining ABCG2 -positive corneal limbal stem cells, the method comprising an induction phase, a differentiation phase and maintenance phase as explained in more detail below, and summarized in Table 1.

TABLE 1
Summary of the producing ABCG2-positive corneal limbal stem cells with one
preferred time schedule and preferred time ranges (duration in days).

| | | Agent/supplement/ | Duration | | Day | |
|---|---|---|---|---|---|---|
| Phase | Step | medium | (days) | | count | Outcome |
| Induction | Optional | Blebbistatin, ROCK | 1 | (0-2) | 1 | Formation of |
| | | inhibitor or other | | | | embryoid bodies |
| | | aggregation-promoting | | | | |
| | | agent | | | | |
| | a) | FGF (preferably FGF-2) + | 1 | (1-7) | 2 | Eye precursor |
| | | TGF-beta inhibitor | | | | cells |
| | | (preferably SB-505124) | | | | |
| | b) | BMP-4 | 2 | (0.5-5) | 4 | |
| Differentiation | c) | Corneal differentiation | 7 | (3-14) | 11 | ABCG2-positive |
| | | medium | | | | limbal stem |
| | | | | | | cells |

| | | | | | |
|---|---|---|---|---|---|
| Maintenance | e) | EGF + at least | At least | 12-67 | Maintenance of |
| | | one Wnt activator | 50 days | | ABCG2-expression |
| | | (preferably CHIR99021 | | | |
| | | and/or R-spondin-1) + | | | |
| | | optionally, noggin | | | |

### Induction Phase

In the induction phase, pluripotent stem cells obtained are induced towards surface ectoderm and eye precursor cells.

The induction phase may be carried out either in a suspension culture or in an adherent culture. If the induction phase is to be carried out in adherent culture, it may be advantageous to use substrates coated with materials such as extracellular matrix (ECM) proteins or combinations thereof as generally known in the art. Preferred ECM proteins include, but are not limited to laminins, collagens, vitronectin, fibronectin, nidogens, and proteoglycans or peptide sequences thereof. Moreover, any coatings suitable for replacing feeder cells may be used to enable the induction phase to be carried out in adherent culture.

In some embodiments, it is preferable to carry out the induction phase in a suspension culture. In such embodiments, a first step of the induction phase comprises forming embryoid bodies from pluripotent stem cells obtained from a feeder-free culture. As used herein, the term “embryoid body” (EB) refers to a three-dimensional cell aggregate. Formation of embryoid bodies can be achieved by various aggregation-promoting methods well known in the art.

As used herein, the term “aggregation-promoting method” refers to any method capable of promoting formation of embryoid bodies from pluripotent stem cells by physical or chemical means.

Formation of embryoid bodies can be achieved, for example, by employing a physical aggregation-promoting method, wherein pluripotent stem cells are cultured in a suspension culture in the presence of non-attachment promoting cell culture surfaces, such as Corning Corning® Costar® Ultra-Low attachment surfaces, or in the presence of one or more agents that prevent cell attachment.

Further non-limiting examples of suitable physical methods of promoting aggregation to achieve EB formation include hanging drop cultures. In such methods, a suspension containing pluripotent stem cells is plated on a lid of a petri dish in regular arrays, inverted lid is then placed over the bottom of a petri dish filled with an appropriate liquid, such as PBS, to prevent the drops from drying out. Eventually, the stem cells fall to the bottom of the hanging drops, and aggregate into a single EB per drop. Also commercial products based on the hanging drop method, such as Perfecta3D® Hanging Drop Plates (3D Biomatrix), may be employed.

Suitable physical aggregation-promoting methods include also methods which are based on multiwell and microfabrication technologies and employ, for example, low-adherence 96-well plates or microwell arrays to induce formation of EBs with a controlled size. Non-limiting examples of such plates or arrays include microwell-patterned poly(dimethylsiloxane) (PDMS) molds with microwells, agarose hydrogel micro-well arrays, as well as commercial AggreWell™ (STEMCELL Technologies) microarray plates.

Also forced aggregation by centrifugation or rotation, or by other physical environments such as microgravity environments may be employed as a physical aggregation-promoting method for obtaining EB formation.

Further suitable aggregation-promoting methods include culturing of cells in presence of agents which aid cell aggregation. Non-limiting examples of such agents include macromolecular crowders, which force cells to closer contact, such as galactose derivatives (e.g. carrageenan), glucose derivatives (e.g. dextran sulfate), polyethylene glycol, and Ficoll®.

Also physical aggregation-promoting methods based on encapsulation or entrapment of pluripotent stem cells in hydrogels, such as methylcellulose, fibrin, hyaluronic acid, dextran, alginate, or agarose, thereby generating individually separated EBs in a semi-solid suspension media, may be used.

Any suitable physical aggregation-promoting method may be used in combination with one or more aggregation promoting chemical agents such as blebbistatin or Rho-associated kinase (ROCK) inhibitors or any other apoptosis inhibiting agents that enhance single cell survival and/or promote stem cell aggregation. Alternatively, cell aggregation may be promoted by chemical means only, for instance by using the chemical agents set forth above.

Conventionally, embryoid bodies may also be formed from pluripotent stem cells by manual separation of adherent colonies or regions of adherent colonies. However, in some embodiments of the present method, manual separation is not a feasible option for obtaining embryoid bodies because pluripotent cells cultured in feeder-free conditions do not spontaneously re-aggregate after manual dissociation to small aggregates or single cells. Addition of one or more aggregation-promoting agents, such as ROCK inhibitors or Blebbistatin, markedly diminish dissociation-induced apoptosis and enables formation of embryoid bodies after dissociation.

Time required for forming embryoid bodies may vary depending on different variables such as the method to be used. Typically the duration of this step is between about 1 hour and about 48 hours, more specifically between about 5 hours and about 24 hours, or overnight, i.e. about 18 hours. However, in some cases, the duration of this step may be even longer than 48 hours. In some embodiments, pluripotent stem cells are cultured in the presence of blebbistatin for about 1 day for the formation of embryoid bodies.

In a second step of the induction phase, embryoid bodies obtained from the first step of the induction phase are subjected to an induction medium comprising active “induction supplements”, i.e. a TGF-beta inhibitor and FGF. If adherent cells are employed (i.e. the step of forming embryoid bodies is omitted), subjecting the cells to the induction medium may be regarded as the first step of the induction phase. Said induction supplements were found to enhance induction of pluripotent stem cells towards eye precursor cells and improve their further differentiation efficiency into clinically valuable corneal limbal stem cells.

In some preferred embodiments, the amount of the TGF-beta inhibitor in the induction medium is from about 1 μM to about 100 μM, preferably from about 1 to about 30 μM, and/or the amount of fibroblast growth factor is from about 1 ng/ml to about 1000 ng/ml, preferably about 2 ng/ml to about 100 ng/ml, and more preferably about 30 ng/ml to about 80 ng/ml.

The present induction medium may be considered to consist of or comprise a basal medium and the present induction supplements. However, further supplements common in the art may be applied. As used herein, the term “common cell culture supplements” refers to ingredients used in practically every cell culture medium including antibiotics, L-glutamine, and serum, serum albumin or a serum replacement, preferably a defined serum replacement.

On the other hand, in some embodiments, the induction medium does not contain ingredients other than the induction supplements, basal medium, antibiotics, L-glutamine, and a defined serum replacement.

In some more specific embodiments, a TGF-beta inhibitor of Formula I or II, and bFGF are used as the induction supplements. In some even more specific embodiments, the induction supplements are SB-505124, and bFGF. In some still even more specific embodiments, SB-505124 is used in a concentration of about 10 μM and bFGF is used in a concentration of about 50 ng/ml.

Any of the aforementioned embodiments may form a basis for additional or alternative embodiments, wherein the induction medium does not comprise any supplements generally known to be inductive for differentiation towards lineages other than eye lineages, including neural differentiation. Such generally known supplements include, but are not limited to, retinoic acid, ascorbic acid, brain-derived neurotrophic factor (BDNF), and glial-derived neurotrophic factor (GDNF). In some preferred embodiments, the induction medium does not contain any Wnt inhibitors.

Time used for inducing the adherent cells or the embryoid bodies with the induction supplements may vary depending on different variables such as the cell line, early differentiation status of the cells, and the specific supplements to be used and concentrations thereof. Typically the duration of this step varies from about 1 day to about 7 days (i.e. from about 22 hours to about 185 hours), more specifically from about 1 day to about 5 days (i.e. from about 22 hours to about 132 hours). In some embodiments, the embryoid bodies are cultured in the presence of induction supplements for about 1 day (i.e. about 22 to 26 hours).

In a next step of the induction phase, the first induction supplements (TGF-beta inhibitor and FGF) are withdrawn and the adherent cells or embryoid bodies obtained from the previous step of the induction phase are cultured in the presence of bone morphogenetic protein 4 (BMP-4) to drive the cells towards surface ectoderm and, concomitantly, to prevent differentiation towards neural lineages. Typical concentrations include from about 1 ng/ml to about 1000 ng/ml, preferably from about 10 ng/ml to about 50 ng/ml, and more preferably about 25 ng/ml.

Time used for inducing the adherent cells or embryoid bodies with BMP-4 may vary depending on different variables such as the concentration of BMP-4. Typically, the duration of this step varies from about 12 hours to about 5 days (i.e. from about 12 hours to about 132 hours), preferably from about 24 hours to about 4 days (i.e. from about 24 hours to about 105 hours), and more specifically 2 days (i.e. from about 43 hours to about 53 hours), and it may or may not involve replacing the culture medium with fresh medium. In some embodiments, this step is carried out for about 2 days, preferably first for about 1 day in medium supplemented with BMP-4, preferably in an amount of about 25 ng/ml, and then about 1 further day in fresh medium also supplemented with BMP-4, preferably in an amount of about 25 ng/ml.

Overall duration of the induction phase may vary depending on different variables. Typically, the duration of the induction phase may vary from a couple of days to several days. A preferred time range is from about 2.5 days to about 18 days, i.e. from about 54 hours to about 475 hours. In some embodiments, preferred overall duration of the induction phase is from about 3 to about 7 days (i.e. from about 65 hours to about 185 hours, or from about 3 days to about 5 days (i.e. from about 65 hours to about 132 hours). More specifically, a preferred overall duration of the induction phase is 4 days, including an optional 1-day step of formation of embryoid bodies. As used herein, the term “about” refers to a variation of about 10 percent of the value specified. Thus, the term “about three days” carries a variation from 65 to 79 hours, while the term “about seven days” carries a variation from 152 to 186 hours, for example. If shorter induction times are used, down-regulation of OCT4 and up-regulation of PAX6 may be weak leading to less efficient differentiation as determined by weak expression of precursor markers PAX6 and p63. Moreover, if longer induction times are used, more neural differentiation can be expected, because human pluripotent stem cells have a known tendency to differentiate towards neural lineages, especially in the presence of basic fibroblast growth factor.

### Aggregation-Promoting Agent

As used herein, the functional term “aggregation-promoting agent” refers to an agent capable of promoting formation of embryoid bodies. Non-limiting examples of aggregation-promoting agents include blebbistatin and Rho-associated kinase (ROCK) inhibitors disclosed in more detail below.

Blebbistatin is a cell-permeable, selective, and reversible inhibitor of nonmuscle myosin II. The name is derived from its ability to inhibit cell blebbing. The actin-myosin based cytoskeleton is a dynamic system essential for cell contraction, motility, and tissue organization. Actin-myosin motors consist of actin filaments and non-muscle myosin II heavy chains that slide along actin filaments, resulting in contraction. The process is triggered by the binding of myosin light chain, which is activated by phosphorylation through kinases, such as Rho-associated kinase (ROCK). Removal from ECM and epithelial cell contacts leaves actin-myosin free to contract, generating altered phenotypes, including excessive cell membrane blebbing and ultimately cell death. Disruption of actin-myosin contraction in individualized human ES cells dramatically improves cell survival and cloning efficiency. Actin-myosin contraction is a downstream target of ROCK regulation. Blebbistatin is readily available in the art.

ROCK inhibitors are cell-permeable, highly potent and selective inhibitors of Rho-associated coiled-coil forming protein serine/threonine kinase (ROCK) family of protein kinases. Non-limiting examples of ROCK inhibitors include chroman 1, Fasudil, Fasudil Hydrochloride, GSK269962A, GSK429286A, H-1152, H-1152 dihydrochloride, Hydroxyfasudil, Hydroxyfasudil hydrochloride, K-115, K-115 free base, LX7101, RKI-1447, ROCK inhibitor(azaindole 1), SAR407899, SAR407899 hydrochloride, SLx-2119, SR-3677, Thiazovivin, and Y-27632, all available e.g. from MedChem Express.

A preferred ROCK inhibitor is Y-27632 (dihydrochloridetrans-4-[(1R)-1-Aminoethyl]-N-4-pyridinylcyclohexanecarboxamide dihydrochloride), which inhibits both ROCK1 and ROCK2 by competing with ATP for binding to the catalytic site. Moreover, it is a potent inhibitor of pluripotent stem cell apoptosis (anoikis), permits survival of dissociated human pluripotent stem cells, improves embryoid body formation in forced-aggregation protocols, and increases the survival of cryopreserved single human ES cells after thawing.

Those skilled in the art can easily determine using various methods readily available in the art, whether or not a given agent has aggregation-promoting activity or not, and whether it is suitable for use in the present method. Non-limiting examples of such methods include visual evaluation of aggregate formation and cell viability assays.

### TGF-beta (TGF-β) Inhibitor

As used herein, with “TGF-beta inhibitor” is referred functionally to a substance capable of inhibiting transforming growth factor β1.

Transforming growth factor β1 (TGF-β1) is a member of a large superfamily of pleiotropic cytokines that are involved in many biological activities, including growth, differentiation, migration, cell survival, and adhesion in diseased and normal states. Nearly 30 members have been identified in this superfamily. These are considered to fall into two major branches: TGFβ/Activin/Nodal and BMP/GDF (Bone Morphogenetic Protein/Growth and Differentiation Factor). They have very diverse and often complementary functions. Some are expressed only for short periods during embryonic development and/or only in restricted cell types (e.g. anti-Mullerian hormone, AMH, Inhibin) while others are widespread during embryogenesis and in adult tissues (e.g. TGFβ1 and BMP4). TGF-β1 is a potent regulator in the synthesis of the extracellular matrix (fibrotic factor) and plays a role in wound healing.

In chemical and structural terms, suitable TGF-beta inhibitory function may be found among proteins and small organic molecules. A man skilled in the art is aware of means for isolating proteins from biological matrixes or producing them i.e. by recombinant techniques.

Compounds exhibiting TGF-beta inhibitory activity may be found by screening. Preferably, a TGF-beta inhibitor is an organic molecule having a relatively low molar mass, e.g. a small molecule having molar mass less than 800 g/mol, preferably less than 500 g/mol. As a general structure, Formula I, a suitable low molar mass TGF-beta inhibitor may be described as:

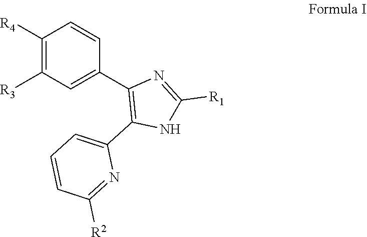

wherein R₁ represents a C₁-C₅ aliphatic alkyl group, carboxylic acid, amide, and R₂ represents a C₁-C₅ aliphatic alkyl, R₃ and R₄ represent aliphatic alkyls including heteroatoms, O or N, which may be linked together to form a 5- or 6-membered hetero ring.

A typical structure comprises a hetero ring having 2 oxygen atoms, when it can be referred to as a small molecule of general formula II:

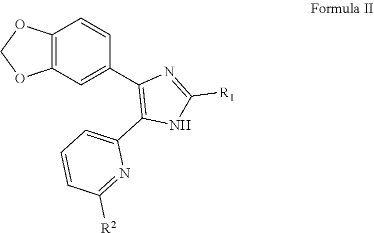

wherein, R₁ represents a C₁-C₅ aliphatic alkyl group, an aromatic carboxylic acid or amide, and R₂ represents a C₁-C₅ aliphatic alkyl.

One non-limiting example of such a TGF-beta inhibitor is 4-[4-(1,3-Benzodioxol-5-yl)-5-(2-pyridinyl)-1H-imidazol-2 -yl]-benzamide also known as SB 431542, which is commercially available from multiple suppliers and marketed as a selective inhibitor of transforming growth factor-β type I receptor (ALK5), ALK4 and ALK7. Another non-limiting example of specific TGF-inhibitors is 2-(5-benzo[1,3]dioxol-5 -yl-2 -tert-butyl-3H -imidazol-4-yl)-6-methylpyridine hydrochloride hydrate, also known as SB 505124.

However, other small molecules exhibiting TGF-beta inhibitory activity or commercially marketed as TGF-inhibitors may be equally suitable in the context of the present invention. When selecting said TGF-beta inhibitor from substances obtainable by chemical synthesis or recombinant production, a defined medium can be provided. It also complies with requirements of xeno-free and serum-free conditions.

Those skilled in the art can easily determine, using various methods readily available in the art, whether or not a given agent has TGF-beta inhibiting activity or not, and whether it is suitable for use in the present method.

### Fibroblast Growth Factor

In the induction medium of the present invention, a fibroblast growth factor is required to contribute to the differentiation. Fibroblast growth factors, or FGFs, are a family of growth factors generally involved in angiogenesis, wound healing, and embryonic development. The FGFs are heparin-binding proteins, and interactions with cell-surface-associated heparan sulfate proteoglycans have been shown to be essential for FGF signal transduction. FGFs are key players in the processes of proliferation and differentiation of wide variety of cells and tissues.

A preferred fibroblast growth factor suitable for use in the present invention is basic FGF (bFGF or FGF-2). However, in some embodiments, other materials, such as certain synthetic small peptides (e.g. produced by recombinant DNA variants or mutants) designed to activate fibroblast growth factor receptors, may be used instead of FGF. Fibroblast growth factors may be included in the serum replacement used as basal medium or they may be added separately to the final cell culture medium according to the present invention.

### Differentiation Phase

The above-described induction phase, preferably but not necessarily carried out in a suspension culture, is followed by a differentiation phase in adherent culture. In the latter phase, eye precursor cells produced in the induction phase are differentiated into ABCG2-positive corneal limbal stem cells.

Since the differentiation phase is to be performed in an adherent culture and the ability to attach to extracellular matrix (ECM) is considered to be important for epithelial cells, it is advantageous to use substrates, such as cell culture plates or bottles, coated with ECM proteins as generally known in the art. Preferred ECM proteins include collagen IV and laminin, preferably laminin-521 and/or laminin-511. More preferably, the cell culture substrate is coated with a mixture of collagen IV and laminin-521 and/or laminin-511, even more preferably with 5 μg/cm² of collagen IV and 0.75 μg/cm² of laminin-521. Other non-limiting examples of suitable coating materials include collagen I, vitronectin, fibronectin, nidogens, proteoglycans, or peptide sequences thereof, commercial attachment and culture substrates comprising the same, such as CELLstart™, and basement membrane extracts, such as Matrigel™ or Geltrex®. Moreover, any coatings suitable for replacing feeder cells may be used in differentiation phase. When xeno-free conditions are desired for human use, the substrate is to be coated with one or more ECM proteins of human or recombinant human origin. Means and methods for coating cell culture substrates are generally available in the art.

Typically, obtaining ABCG2-positive corneal limbal stem cells requires culturing eye precursor cells under the present corneal differentiation conditions for about 3 to about 14 days, preferably for about 7 days. In some preferred embodiments, ABCG2-positive corneal limbal stem cells are obtained by carrying out the induction phase for about 2.5 days to about 18 days followed by the corneal differentiation phase for about 3 to 14 days. In some even more preferred embodiments, ABCG2-positive corneal limbal stem cells are obtained by carrying out the induction phase for about 4 days followed by the corneal differentiation phase for about 6 to 7 days. The most pure ABCG2-positive cell population can be obtained after a differentiation stage of this length. Shorter differentiation time yields more heterogeneous cell populations, while longer differentiation time results in concomitant loss of ABCG2 expression and emergence of p63 expression followed by terminal maturation towards corneal epithelial cells. Non-limiting examples of markers for maturated corneal epithelium include CK12 and CK3.

As used herein, the term “corneal differentiation medium” refers to a cell culture medium supporting differentiation of cells towards the corneal lineage, preferably ABCG2-positive corneal limbal stem cells. Those skilled in the art can easily determine, using various methods readily available in the art, whether or not a given cell culture medium may be regarded as a corneal differentiation medium and, thus, whether it is suitable for use in the present differentiation phase.

In accordance with the above, a suitable culture medium for use in the differentiation phase may be, for instance, any corneal medium such as CnT-30 which is commercially available from CELLnTECH, or any supplemental hormonal epithelial medium (SHEM) suitable for culturing corneal epithelial cells. In some other embodiments, the differentiation medium may be composed by adding ingredients such as one or more differentiation supplements selected from the group consisting of epidermal growth factor (EGF), hydrocortisone, insulin, isoproterenol and tri-iodo-thyronine, into any suitable basal medium. In some embodiments, the corneal differentiation medium does not contain ingredients other than said one or more differentiation and maturation supplements, basal medium, antibiotics, L-glutamine, and a defined serum replacement. In other words, in some embodiments, the corneal differentiation medium does not contain any of the following ingredients: a TGF-beta inhibitor, a fibroblast growth factor, and BMP-4, or any functionally equivalent agents. In some further embodiments, said corneal differentiation medium does not comprise a Wnt-inhibitor either.

Any of the aforementioned embodiments may form a basis for additional or alternative embodiments, wherein the differentiation medium does not comprise any supplements, which are generally known to cause differentiation towards cell lineages other than eye lineages, including neural differentiation. Such generally known supplements include, but are not limited to, retinoic acid, ascorbic acid, BDNF, and GDNF.

### Maintenance Phase

The above-described differentiation phase is followed by a maintenance phase in adherent culture. In this phase, the ABCG2-positive phenotype of the corneal limbal stem cells obtained in the differentiation phase is maintained.

Suitable culture media for use in the maintenance phase may be, for instance, any corneal medium or any supplemental hormonal epithelial medium (SHEM) available in the art and suitable for culturing corneal epithelial cells. Non-limiting examples include CnT-07 and CnT-30 which are commercially available from CELLnTE CH, and XFDM—which is disclosed in Hongisto et al. 2017, Stem Cells. In some other embodiments, the maintenance medium may be composed by adding at least epidermal growth factor (EGF) and at least one Wnt activator as differentiation supplements into any suitable basal medium. Preferred Wnt activators include Glycogen synthase kinase 3 (GSK3) inhibitors such as CHIR99021, and R-spondin or its substitutes such as RS-246204. A further preferred maintenance supplement is Noggin. In some embodiments, the maintenance medium may further comprise, hydrocortisone, insulin, isoproterenol, and tri-iodo-thyronine. Alternatively or in addition, in some embodiments, the maintenance medium does not contain ingredients other than said maintenance supplements, basal medium, antibiotics, L-glutamine, and a defined serum replacement. Accordingly, in some embodiments, the medium does not contain any of the following ingredients: a TGF-beta inhibitor, a fibroblast growth factor, and BMP-4, or any functionally equivalent agents.

In some embodiments, ABCG2-positive phenotype may be maintained for at least 50 days and over 6 passages, as well as over cryostorage.

### Epidermal Growth Factor

In the differentiation medium of the present invention, epidermal growth factor is required for maintaining the ABCG2 expression of corneal limbal stem cells.

In some preferred embodiments, the amount of EGF in the differentiation medium is from about 1 ng/ml to about 150 ng/ml, preferably from about 1 ng/ml to about 100 ng/ml, and more preferably from about 5 ng/ml to about 50 ng/ml.

In some embodiments, other materials, such as certain synthetic small peptides (e.g. produced by recombinant DNA variants or mutants) designed to activate epidermal growth factor receptors (EGFR), may be used instead of EGF.

EGF may be provided in a serum replacement, basal medium or it may be added separately to the final cell culture medium according to the present invention.

### Wnt Activators

The Wnt family of protooncogenes consists of at least 16 known members which encode secreted signaling proteins that are involved in oncogenesis and several other developmental processes, such as regulation of cell fate and embryogenesis. As used herein, the term “Wnt activator” refers to a substance capable of activating Wnt signaling pathway. Both protein and small molecular Wnt activators are known in the art.

Glycogen synthase kinase 3 (GSK3) inhibitors are an exemplary class of preferred Wnt activators for use in the present invention. CHIR99021, which is commercially available from multiple providers, is the most selective inhibitor of GSK3 and, thus, a particularly preferred Wnt activator for use in the present invention. Further GSK3 inhibitors include, but are not limited to, SB-216763, BIO(6-bromoindirubin-3′-oxime), LY2090314 and lithium chloride, all of which are commercially available.

In some preferred embodiments, the amount of the GSK3 inhibitor, such as CHIR99021, in the differentiation medium is from about 1 μM to about 15 μM, from about 1 μM to about 10 04, from about 1 μM to about 5 μM, preferably about 3 μM.

Another class of Wnt activators suitable for use in the present invention is the R-spondin protein family, the four members of which, designated as R-spondin-1, R-spondin-2, R-spondin-3 and R-spondin-4, are secreted agonists of the canonical Wnt/β-catenin signaling pathway.

In some embodiments, the Wnt activator is R-spondin-1. Preferred concentration ranges include from about 100 ng/ml to about 2 μg/ml, from about 500 ng/ml to about 2 μg/ml, preferably about 1 μg/ml.

RS-246204 is a small molecule R-spondin-1 substitute, which is also suitable for use in the present invention. Preferred concentration ranges include from about 6.25 μM to about 200 μM, and from about 25 μM to about 50 μM.

In some embodiments, the differentiation supplements may comprise at least one GSK3 inhibitor, such as CHIR99021, and at least one R-spondin, such as R-spondin-1. In some other embodiments, increasing the amount of said at least one GSK3 inhibitor may be used to replace the presence of said at least one R-spondin, and vice versa.

Those skilled in the art can easily determine using various methods readily available in the art, whether or not a given agent has Wnt-activating properties or not, and whether it is suitable for use in the present method. A compound can be tested for its ability to act as Wnt activators e.g. by a commercial test kit, LEADING LIGHT® Wnt Reporter Assay Starter Kit available from Enzo.

### Noggin

Noggin, also known as NOG, is a protein that is involved in the development of many body tissues, including nerve tissue, muscles, and bones.

In some embodiments of the present invention, the differentiation medium may further comprise or be supplemented with Noggin to support ABCG2 expression of the corneal limbal stem cells.

Typically, the amount of Noggin the differentiation medium may vary from about 10 ng/ml to about 500 ng/ml, and from about 10 ng/ml to about 100 ng/ml, and be for example about 100 ng/ml.

LDN-193189 is a small molecule Noggin substitute, which is also suitable for use in the present invention. Preferred concentration ranges include, for example, from about 0.01 μM to about 1 μM. Further Noggin substitutes include, but are not limited to, dorsomorphin analogues, such as DMH1.

### Method of Maintaining ABCG2-Positive Phenotype of Corneal Limbal Stem Cells

In addition to the method of producing ABCG2-positive corneal limbal stem cells described above, the present invention also provides a method of maintaining ABCG2-positive phenotype of corneal limbal stem cells.

In this method, the corneal limbal stem cells whose ABCG2-positive phenotype is to be maintained may be any ABCG2-positive corneal limbal stem cells including, but not limited to, primary corneal limbal stem cells or corneal limbal stem cells produced by the above-described differentiation method.

The method of maintaining the ABCG2-positive phenotype of corneal limbal stem cells comprises culturing ABCG2-positive phenotype of corneal limbal stem cells in a culture medium comprising EGF and at least one Wnt activator. In some embodiments, the medium comprises EGF and at least one GSK3 inhibitor (such as CHIR99021). In some other embodiments, the medium comprises EGF and R-spondin (such as R-spondin-1) or a substitute thereof (such as RS-246204). In some further embodiments, the medium comprises EGF, at least one GSK3 inhibitor (such as CHIR99021) and R-spondin (such as R-spondin-1) or a substitute thereof (such as RS-246204). In each of these embodiments, the medium may further comprise Noggin. Preferred concentration ranges of these supplements described above with respect to the method of producing ABCG2 -positive corneal limbal stem cells, apply also to the method of maintaining the ABCG2-positive phenotype of corneal limbal stem cells.

In some embodiments, ABCG2-positive phenotype may be maintained for at least 50 days and over 6 passages, as well as over cryostorage. Any embodiments described in relation to the maintenance phase of the present method of producing ABCG2-positive corneal limbal stem cells apply to the present method of maintaining ABCG2-positive phenotype of corneal limbal stem cells, and vice versa, unless otherwise indicated.

### General Features of Culture Conditions and Media

Any culture medium may be considered to consist of basal medium and supplements. In the present induction medium, the essential supplements are first a TGF-beta inhibitor and FGF and then BMP-4; whereas in the present maintenance medium, required supplements comprise EGF and at least one Wnt activator. However, in the context of culture media, further supplements common in the art may be applied, unless they are known to direct differentiation towards tissues other than eye tissues. When referring to components of a medium, the term includes both supplements and ingredients to the basal medium.

When in use or when ready for use, the present culture media comprise appropriate essential supplements set forth above. However, according to common practice in the field, the ingredients for a medium may be provided as a concentrate comprising said components or a set of vials from which suitable combination is prepared prior to use in a laboratory according to instructions provided. Often, culture medium is diluted and prepared to the final composition immediately before use. Therefore, it is understood that any stock solution or preparation kit suitable for use in such immediate preparation may be used for obtaining cell culture media to be used in the present method. For example, for the preparation of an induction medium set forth above, a cell culture kit comprising the TGF-beta inhibitor and the fibroblast growth factor, each in a separate container or in any combinations thereof, as supplements, and optionally other components, such as basal medium or supplies for preparation thereof, may be employed.

As referred here, “culture medium” or “cultivation medium” refers broadly to any liquid or gel formulation designed to support the growth of microorganisms, cells or small plants. When referring to formulation designed for cell maintenance and growth, term “cell culture medium” is used. In the art, expressions such as induction medium, growth medium, differentiation medium, maturation medium etc. can be considered as subspecies to the general expression “culture medium”. Those skilled in the art are familiar with the basic components necessary to maintain and nourish living cells in or on the culture medium, and commercial basic media are widely available. Typically such basic components are referred to as “basal medium”, which contains necessary amino acids, minerals, vitamins and organic compounds. Generally, a basal medium may be combined from isolated and pure ingredients. If desired, basal medium may be supplemented with substances contributing to special features or functions of culture medium. Very common supplements include antibiotics, which are used to limit growth of contaminants, L-glutamate and serum, serum albumin or a serum replacement. Those skilled in the art are familiar with both necessary or optional culture medium component and concentrations thereof.

For use in the present method and its various embodiments, the basal medium may be any stem cell culture medium in which stem cells can effectively be differentiated. Non-limiting examples of suitable basal media include KnockOut Dulbecco's Modified Eagle's Medium (KO-DMEM), Dulbecco's Modified Eagle's Medium (DMEM), Minimal Essential Medium (MEM), Basal Medium Eagle (BME), RPMI 1640, F-10, F-12, Glasgow's Minimal Essential Medium (G-MEM), Iscove's Modified Dulbecco's Medium and any combinations thereof. In some preferred embodiments, RegES medium altered by omitting retinol, bFGF and activin A (referred to herein as RegESbasic and disclosed in Vaajasaari et al., Mol Vis. 2011; 17:558-75) is used as a basal medium. In some more preferred embodiments, RegESbasic is used as a basal medium in the present induction medium.

For better clinical acceptance, all culture media to be used in the present method and its various embodiments are preferably substantially xeno-free, substantially serum-free, or substantially defined, more preferably combinations of these, and most preferably substantially xeno-free, substantially serum-free, and substantially defined at the same time. With “substantially” is meant herein that unintentional traces are irrelevant, and what is under clinical or laboratory regulations considered and accepted as xeno-free, serum-free or defined, applies here as well.

As used herein the term “xeno-free” refers to absence of any foreign material or components. Thus, in case of human cell culture, this refers to conditions free from non-human animal components. In other words, when xeno-free conditions are desired for production of corneal cells for human use, all components of any cell culture media must be of human or recombinant origin.

Traditionally, serum, especially fetal bovine serum (FBS) has been valued in cell cultures providing essential growth and survival components for in vitro cell culture of eukaryotic cells. It is produced from blood collected at commercial slaughterhouses from cattle bred to supply meat destined for human consumption. “Serum free” indicates that the culture medium contains no serum, either animal or human. Defined medium is valuated when there are contradictions for use of undefined media, e.g. “conditioned medium”, which refers to spent media harvested from cultured cells containing metabolites, growth factors, and extracellular matrix proteins secreted into the medium by the cultured cells. Undefined media may be subject to considerable dissimilarities due to natural variation in biology. Undefined components in a cell culture compromise the repeatability of cell model experiments e.g. in drug discovery and toxicology studies. Hence, “defined medium” or “defined culture medium” refers to a composition, wherein the medium has known quantities of all ingredients. Typically, serum that would normally be added to culture medium for cell culture is replaced by known quantities of serum components, such as, e.g., albumin, insulin, transferrin and possibly specific growth factors (e.g., basic fibroblast growth factor, transforming growth factor or platelet-derived growth factor).

A chemically defined medium is a growth medium suitable for in vitro cell culture of human or animal cells in which all of the chemical components are known. A chemically defined medium is entirely free of animal-derived components and represents the purest and most consistent cell culture environment. By definition, chemically defined media cannot contain fetal bovine serum, bovine serum albumin or human serum albumin as these products are derived from bovine or human sources and contain complex mixes of albumins and lipids.

Chemically defined media differ from serum-free media in that bovine serum albumin or human serum albumin is replaced with either a chemically defined recombinant version (which lacks the albumin associated lipids) or a synthetic chemical, such as the polymer polyvinyl alcohol, which can reproduce some of the functions of BSA/HSA. The next level of defined media, below chemically defined media is protein-free media. These media contain animal protein hydrolysates and are complex to formulate although are commonly used for insect or CHO cell culture.

According to some embodiments, the present media comprises a xeno-free serum replacement formulation. A defined xeno-free serum replacement formulation or composition may be used to supplement any suitable basal medium for use in in vitro derivation, maintenance, proliferation, or differentiation of stem cells. Said serum replacement may be used to supplement either serum-free or serum-containing basal mediums, or any combinations thereof. When xeno-free basal medium is supplemented with a xeno-free serum replacement, the final culture medium is xeno-free as well. One example is described in Rajala et al. 2010, which is incorporated here as reference, describing a xeno-free serum replacement applicable in the context of the present invention. Another non-limiting example of a serum replacement is KnockOut™ Serum Replacement (Ko-SR), and xeno-free version KnockOut™ SR XenoFree CTS™ both commercially available from Life Technologies.

### Therapeutic Use

The present invention also provides a method of treating an eye disease, such as limbal stem cell deficiency (LSCD), in a subject in need thereof. The method comprises administering an efficient amount of ABCG-positive corneal limbal stem cells produced or maintained in accordance with the present invention to said subject.

In accordance with the above, the present invention also provides ABCG2-positive corneal limbal stem cells produced or maintained in accordance with the present invention for use in treating an eye disease such as LSCD.

As used herein, the term “subject” refers to any mammals, preferably humans.

The ABCG2-positive corneal limbal stem cells for use in therapy may be either allogenic or autologous.

As used herein, the term “efficient amount” refers to an amount of ABCG2-positive corneal limbal stem cells by which harmful effects of the eye disease are, at a minimum, ameliorated. In view of the fact that cultures of limbal stem cells, wherein p63 positive cells represent even as little as only 3% of the cell population, have been reported to provide therapeutic success rate of 78% (Rama et al. 2010, N Engl J Med, 363:147-55), a population of corneal limbal stem cells comprising as little as only 3% or more of ABCG2-positive cells may also be considered clinically relevant and thus encompassed by the term “efficient amount”. Preferably, however, an efficient amount of ABCG2-positive corneal limbal stem cells refers to a cell population, wherein ABCG2-positive cells represent at least 65%, preferably at least 75%, most preferably at least 90% of the total cell population.

As used herein, the term “treatment” or “treating” is intended to include the administration of the present ABCG2-positive corneal limbal stem cells to a subject for purposes which may include ameliorating, lessening, inhibiting, or curing the disease.

### Numbered Exemplary Embodiments of the Invention

1. A method of maintaining ABCG2 -positive phenotype of corneal limbal stem cells, wherein the method comprises culturing ABCG2-positive corneal limbal stem cells in a culture medium comprising EGF and at least one Wnt activator.

2. The method according to embodiment 1, wherein the amount of EGF is from about 1 ng/ml to about 150 ng/ml, preferably from about 1 ng/ml to about 100 ng/ml, and more preferably from about 15 ng/ml to about 50 ng/ml.

3. The method according to embodiment 1 or 2, wherein the Wnt activator is selected from the group consisting of GSK3 inhibitors and proteins of R-spondin family.

4. The method according to embodiment 3, wherein the GSK3 inhibitor is selected from the group consisting of CHIR99021, SB-216763, BIO(6-bromoindirubin-3′-oxime), LY2090314 and lithium chloride.

5. The method according to embodiment 4, wherein GSK3 inhibitor is CHIR99021.

6. The method according to embodiment 5, wherein the amount of CHIR99021 is from about 1 μM to about 15 μM, from about 1 μM to about 10 μM, from about 1 μM to about 5 μM, or about 3 μM.

7. The method according embodiment 3, wherein the protein of R-spondin family is selected from the group consisting of R-spondin-1 and its supplement RS-246204, R-spondin-2, R-spondin-3 and R-spondin-4.

8. The method according to embodiment 7, wherein protein of R-spondin family is R-spondin-1.

9. The method according to embodiment 8, wherein the amount of R-spondin-1 is from about 100 ng/ml to about 2 μg/ml, from about 500 ng/ml to about 2 μg/ml, or about 1 μg/ml.

10. The method according to embodiment 7, wherein the amount of RS-246204 from about 6.25 μM to about 200 μM, or from about 25 μM to about 50 μM.

11. The method according to any one of embodiments 1-10, wherein the culture medium further comprises noggin or its supplement selected from the group consisting of LDN-193189 and dorsomorphin analogues, such as DMH1.

12. The method according to embodiment 11, wherein the amount of Noggin is from about 10 ng/ml to about 500 ng/ml, from about 10 ng/ml to about 100 ng/ml, or about 100 ng/ml.

13. The method according to embodiment 11, wherein the amount of LDN-193189 is from about 0.01 μM to about 1 μM.

14. The method according to any one of embodiments 1-13, wherein the ABCG2-positive corneal limbal stem cells are ABCG2-positive primary corneal limbal stem cells or pluripotent stem cell-derived ABCG2-positive corneal limbal stem cells.

15. The method according to embodiment 14, wherein the cells are human cells.

16. A method of producing ABCG2-positive corneal limbal stem cells, the method comprising:

a) providing pluripotent stem cells;

b) culturing said cells in a cell culture medium comprising a TGF-beta inhibitor and a fibroblast growth factor (FGF), preferably basic FGF;

c) withdrawing the TGF-beta inhibitor and the FGF, and culturing the cells obtained in step b) in a cell culture medium comprising bone morphogenetic protein 4 (BMP-4) thereby producing eye precursor cells;

d) culturing said eye precursor cells in a corneal differentiation medium thereby producing ABCG2-positive corneal limbal stem cells; and

e) culturing said ABCG2-positive corneal limbal stem cells in a maintenance cell culture medium comprising EGF and at least one Wnt activator, thereby maintaining ABCG2-positive corneal limbal stem cell phenotype obtained via steps a-d).

17. A method of producing ABCG2-positive corneal limbal stem cells, the method comprising:

a) culturing pluripotent stem cells in a cell culture medium comprising a TGF-beta inhibitor and a fibroblast growth factor (FGF), preferably basic FGF;

b) withdrawing the TGF-beta inhibitor and the FGF, and culturing the cells obtained in step a) in a cell culture medium comprising bone morphogenetic protein 4 (BMP-4) thereby producing eye precursor cells;

c) culturing said eye precursor cells in a corneal differentiation medium thereby producing ABCG2-positive corneal limbal stem cells; and

d) culturing said ABCG2-positive corneal limbal stem cells in a maintenance cell culture medium comprising EGF and at least one Wnt activator, thereby maintaining ABCG2-positive corneal limbal stem cell phenotype obtained via steps a-e).

18. The method according to embodiment 16 or 17, wherein the TGF-beta inhibitor is selected from TGF-beta inhibitors having molar mass of less than 800 g/mol, preferably less than 500 g/mol.

19. The method according to embodiments 18, wherein the TGF-beta inhibitor is selected from organic molecules according to Formula I

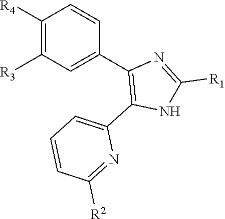

wherein R₁ represents an C₁-C₅ aliphatic alkyl group, carboxylic acid, amide, and R₂ represents an C₁-C₅ aliphatic alkyl, R₃ and R₄ represent aliphatic alkyls including heteroatoms, O or N, which may be linked together to form a 5- or 6-member heteroring.

20. The method according to any one of embodiments 16-18, wherein fibroblast growth factor is selected from basic FGF and synthetic small peptides exhibiting fibroblast growth factor-like activity.

21. The method according to any one of embodiments 16-20, wherein the amount of TGF-beta inhibitor is from 1 μM to 100 μM, preferably from 1 to 30 μM.

22. The method according to any one of embodiments 16-21, wherein the amount of fibroblast growth factor is from 1 ng/ml to about 1000 ng/ml, preferably about 2 ng/ml to about 100 ng/ml, and more preferably about 30 ng/ml to about 80 ng/ml.

23. The method according to any one of embodiments 16-22, wherein the amount of BMP-4 is 1 ng/ml to 1000 ng/ml, preferably about 10 ng/ml to 50 ng/ml, and more preferably 25 ng/ml.

24. The method according to any one embodiment 16-23, wherein the amount of EGF is from about 1 ng/ml to about 150 ng/ml, preferably from about 1 ng/ml to about 100 ng/ml, and more preferably from about 15 ng/ml to about 50 ng/ml. 25. The method according to any one of embodiments 16-24, wherein the Wnt activator is selected from the group consisting of GSK3 inhibitors and proteins of R-spondin family.

26. The method according to embodiment 25, wherein the GSK3 inhibitor is selected from the group consisting of CHIR99021, SB-216763, BIO(6-bromoindirubin-3′-oxime), LY2090314 and lithium chloride.

27. The method according to embodiment 26, wherein GSK3 inhibitor is CHIR99021.

28. The method according to embodiment 27, wherein the amount of CHIR99021 is from about 1 μM to about 15 μM, from about 1 μM to about 10 μM, from about 1 μM to about 5 μM, or about 3 μM.

29. The method according embodiment 25, wherein the protein of R-spondin family is selected from the group consisting of R-spondin-1 and its supplement RS-246204, R-spondin-2, R-spondin-3 and R-spondin-4.

30. The method according to embodiment 29, wherein protein of R-spondin family is R-spondin-1.

31. The method according to embodiment 30, wherein the amount of R-spondin-1 is from about 100 ng/ml to about 2 μg/ml, from about 500 ng/ml to about 2 μg/ml, or about 1 μg/ml.

32. The method according to embodiment 29, wherein the amount of RS-246204 from about 6.25 μM to about 200 μM, or from about 25 μM to about 50 μM.

33. The method according to any one of embodiments 16-32, wherein the maintenance culture medium further comprises noggin or its supplement selected from the group consisting of LDN-193189 and dorsomorphin analogues, such as DMH1.

34. The method according to embodiment 33, wherein the amount of noggin is from about 10 ng/ml to about 500 ng/ml, from about 10 ng/ml to about 100 ng/ml, or about 100 ng/ml.

35. The method according to embodiment 33, wherein the amount of LDN-193189 is from about 0.01 μM to about 1 μM.

36. The method according to any one of embodiments 16-35, wherein the pluripotent stem cells are selected from induced pluripotent stem (iPS) cells and embryonic stem (ES) cells, with the proviso that if human embryonic stem (hES) cells are used, the method does not include the destruction of human embryos.

37. The method according to any one of embodiments 16-36, wherein the cells are human cells.

38. The method according to embodiment 16 and embodiments 18-37 when referring to it, wherein step a) comprises forming embryoid bodies from said pluripotent stem cells.

39. The method according to embodiment 17 and embodiments 18-37 when referring to it, wherein step a) is preceded by a step comprising forming embryoid bodies from said pluripotent stem cells.

40. The method according to embodiment 38 or 39, wherein said forming of embryoid bodies is carried out by a physical or chemical method, preferably selected from the group consisting of culturing cells in the presence of attachment-preventing agents, culturing cells in hanging drops, microfabrication techniques, forced aggregation e.g. by centrifugation, and culturing cells in the presence of one or more aggregation-promoting agents such as macromolecular crowders, blebbistatin and ROCK inhibitors.

41. The method according to embodiment 16 and embodiments 18-40 when referring to it, wherein said culturing in steps d) and e) is carried out on a substrate coated at least with collagen IV and laminin, preferably laminin-521 and/or laminin-511.

42. The method according to embodiment 17 and embodiments 18-40 when referring to it, wherein said culturing in steps c) and d) is carried out on a substrate coated at least with collagen IV and laminin, preferably laminin-521 and/or laminin-511.

43. The method according to any one of the preceding embodiments, which is performed in substantially xeno-free, substantially serum-free and/or defined conditions.

44. The method according to embodiment 16 and embodiments 18-43 when referring to it, wherein the duration of step b) is carried out for 1-7 days, preferably 1 day.

45. The method according to embodiment 17 and embodiments 18-43 when referring to it, wherein the duration of step a) is carried out for 1-7 days, preferably 1 day.

46. The method according to embodiment 16 and embodiments 18-43 when referring to it, wherein the duration of step c) is carried out for 0.5-5 days, preferably 2 days.

47. The method according to embodiment 17 and embodiments 18-43 when referring to it, wherein the duration of step b) is carried out for 0.5-5 days, preferably 2 days.

48. The method according to embodiment 16 and embodiments 18-43 when referring to it, wherein the duration of step d) is carried out for 3-14, preferably 6-7 days, more preferably 7 days.

49. The method according to embodiment 17 and embodiments 18-43 when referring to it, wherein the duration of step c) is carried out for 3-14, preferably 6-7 days, more preferably 7 days.

50. The method according to embodiment 16 and embodiments 18-43 when referring to it, wherein the duration of step e) is carried out for any desired number of days, such as less than 50 days or at least 50 days.

51. The method according to embodiment 17 and embodiments 18-43 when referring to it, wherein the duration of step d) is carried out for any desired number of days, such as less than 50 days or at least 50 days.

52. ABCG2-positive corneal limbal stem cells, whose ABCG2-positive phenotype has been maintained by the method of any one of embodiments 1-15 or which have been produced by the method of any one of embodiments 16-51, for use in treating an eye disease, preferably limbal stem cell deficiency (LSCD).

It will be obvious to a person skilled in the art that, as technology advances, the inventive concept can be implemented in various ways. The invention and its embodiments are not limited to the examples described below but may vary within the scope of the claims.

### EXAMPLE 1

### Examination of Differentiation of Human Pluripotent Stem Cells into Corneal Limbal Epithelial Stem Cells Reveals a Subsequent Emergence of Two Distinct Cell Populations Expressing ABCG2 or ΔNp63α

These experiments were carried out in order to analyze the differentiation hierarchy along the differentiation process from human pluripotent stem cells into corneal limbal epithelial stem cells using the differentiation method disclosed in WO 2018/037161.

Materials and methods

### Differentiation Protocol of WO 2018/037161

Three genetically different in-house derived hPSC lines, hESC line Regea08/017 and Regea11/013 (Skottman et al. 2010) and hiPSC line UTA.04607.WT were used in this study. Human PSC cultures were routineously maintained in serum- and feeder cell-free conditions and differentiated towards the corneal epithelial lineage as described previously in Hongisto et al. 2017 Stem Cell Res and Hongisto et al 2018, JoVE. In brief, colonies of undifferentiated hPSCs were enzymatically dissociated to single cell suspension and transferred into defined XF-Ko-SR medium on low-attachment well plates, for the induction in suspension culture. To support the formation of embryoid bodies (EBs) during the first day, 5 μM blebbistatin (Sigma-Aldrich) was added to XF-Ko-SR. During the following three day induction period as EBs, XF-Ko-SR medium was first supplemented with 10 μM SB-505124 and 50 ng/ml human basic fibroblast growth factor (bFGF; PeproTech Inc., Rocky Hill, N.J.) for one day, and with 25 ng/ml bone morphogenetic protein (BMP)-4 (PeproTech Inc.) for two days. After induction, the EBs were transferred onto 0.5 μg/cm² recombinant laminin-521 (LN-521, Biolamina, Sweden) and 5 μg/cm² human placental collagen Type IV (Col IV, Sigma-Aldrich) coated wells in defined commercial CnT-30 corneal differentiation medium (CELLnTEC Advanced Cell Systems AG, Bern, Switzerland) for further epithelial differentiation. In standard differentiation, adherent cultures were thereafter maintained in CnT-30, changing media three times a week until analysis or cryopreservation. Representative images of cell morphology were captured with Nikon Eclipse TE2000-S phase contrast microscope (Nikon Instruments Europe).

Protein Expression Profiling during the Differentiation Protocol with Immunofluorescence

Regea08/017 and UTA.04607.WT undifferentiated (UD) human pluripotent stem cells (hPSCs) and hPSC-derived LSCs were employed for full characterization in time points of 7, 9, 11, 14, 17, 21 and 24 days (including 4 day induction period) for their expression of OCT-3/4, PAX-6, ABCG2, p63α, ΔNp63, CK-15, CK-14 and CK-12 using immunofluorescence labeling (IF). Cytospin samples for quantifying the expression of OCT-3/4, ABCG2, p63α, ΔNp63, CK-15 and CK-14 were prepared at time points of 10 and 24 days. Prior to staining the cells were fixed with 4% paraformaldehyde (PFA, Sigma-Aldrich) 15-20 min at room temperature (RT). Basic IF protocol using primary and secondary antibodies (Table 1) was performed essentially as described previously in Mikhailova et al 2014. Raw images of the stained cells were captured with Olympus IX51 fluorescence microscope and ImageJ Image Processing and Analysis software (https://imagej.nih.gov/ij/) was used for cell counting analysis. IF characterizations and cytospin quantifications were repeated two to three times for both lines, using cells from individual differentiation batches. Similar protein expression profile of hESC line Regea11/013 was subsequently confirmed by IF characterization at days 11 and 24.

TABLE 1

| Antibody | Host | Manufacturer | #Cat. no | Dilution |
|---|---|---|---|---|
| Primary: | | | | |
| OCT3/4 | goat | R&D Systems | AF1759 | 1:200 |
| PAX6 | rabbit | Sigma-Aldrich | HPA030775 | 1:100 |
| ABCG2 | mouse | Millipore | MAB4155 | 1:200 |
| p63α | rabbit | Cell Signaling | 4892S | 1:200 |
| | | Technologies | | |
| ΔNp63 | mouse | BioCare Medical | ACI3066A | 1:100 |
| CK14 | mouse | R&D Systems | MAB3164 | 1:300 |
| CK15 | mouse | Thermo Fischer | MS-1068-P0 | 1:300 |
| | | Scientific | | |
| CK12 | mouse | Santa Cruz | SC-17099 | 1:200 |
| | | Biotechnologies | | |
| Secondary: | | | | |
| Anti-mouse A488 | goat | Molecular Probes | A-21042 | 1:800 |
| Anti-rabbit A488 | donkey | Molecular Probes | A-21206 | 1:800 |
| Anti-goat A488 | donkey | Molecular Probes | A-11055 | 1:800 |
| Anti-mouse A568 | donkey | Molecular Probes | A-10037 | 1:800 |
| Anti-rabbit A568 | donkey | Molecular Probes | A-10042 | 1:800 |
| Anti-goat A568 | donkey | Molecular Probes | A-11057 | 1:800 |
| Anti-mouse A647 | donkey | Molecular Probes | A-31571 | 1:800 |
| Anti-rabbit A647 | donkey | Molecular Probes | A-31573 | 1:800 |
| Anti-goat A647 | donkey | Abcam | ab150131 | 1:800 |

### Fluorescence Activated Cell Sorting (FACS) Analysis for ABCG2

For labeling with FACS antibodies, the cells were first enzymatically dissociated and washed with pre-chilled FACS wash buffer containing 0.5% bovine serum albumin (BSA, Sigma-Aldrich) and 2 nM EDTA (Gibco, Thermo Fisher Scientific) in DPBS (Lonza). Counted cells were divided 2-10×10⁵ cells per sample to 5 ml tubes. Optimized volumes of appropriate antibodies were added to a 100 μl sample volume and incubated 20 min on ice, protected from light. Lastly, the samples were washed twice, resuspended in the wash buffer, and stored on ice until the analysis. APC-conjugated monoclonal mouse anti-human CD338 (ABCG2) antibody, clone 5D3 (BD Pharmingen, #561451) was used to label the cells (3 μl per sample). APC-conjugated mouse IgG2b κ antibody (BD Pharmingen, #555745) served as appropriate isotype control, and unstained negative samples were prepared for gating the populations. FACS analysis was carried out using BD FACSAria™ Fusion cell sorter (BD Biosciences, San Jose, Calif., USA), and the analyses from individual differentiation batches were repeated at least three times for both Regea08/017 and UTA.04607.WT lines. For each sample, 10 000 events were recorded.

### Quantitative RT-PCR for ABCG2

Total RNA was extracted from the pelleted cell samples using RNeasy Mini Kit (Qiagen, Thermo Fisher Scientific) and RNA concentration of each sample was determined with NanoDrop-1000 spectrophotometer (NanoDrop Technologies). 400 ng total RNA from each sample were used for cDNA synthetization using High-Capacity cDNA Reverse Transcription Kit (Applied Biosystems, Thermo Fisher Scientific). The resulting cDNA samples were analyzed for their ABCG2 mRNA with qPCR, using sequence-specific TaqMan Gene Expression Assay (#HS01053790_m1, Applied Biosystems). GAPDH (Hs99999905_m1) was used as a housekeeping gene. All samples and controls were run as triplicate reactions with the 7300 Real-Time PCR system (Applied Biosystems). Results were analyzed using the -2ΔΔCt method (Livak and Schmittgen, 2001) and presented as fold change in gene expression normalized to GAPHD and relative to the undifferentiated control (UD-hPSCs).

### Results

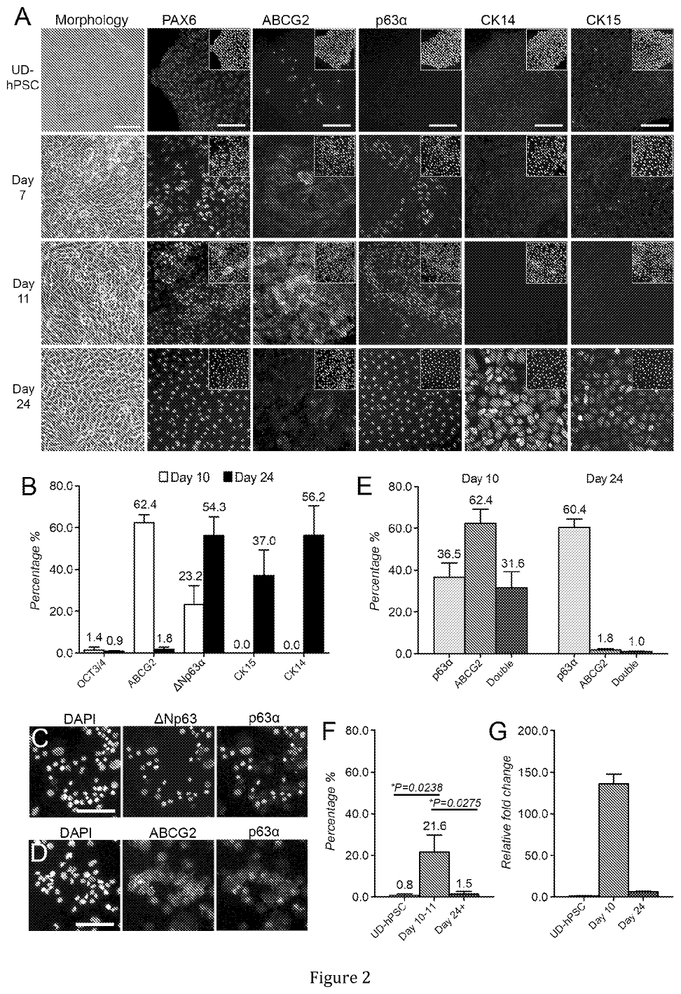

To investigate the protein expression in different stages during the differentiation of hPSC towards LSCS, two genetically distinct feeder-free-cultured hPSCs lines, hESC line Regea08/017 and hiPSC line UTA.04607.WT, were guided towards the corneal lineage using an established two-step protocol (Hongisto et al. 2017 Stern Cell Res Ther). Along the differentiation process, changes in the expression pattern for OCT3/4 as well as several acknowledged LSC- and mature corneal epithelium related marker proteins were extensively characterized using immunofluorescence labeling (IF). Eight IF analysis time points included day 0 (UD-hPSCs), 7, 9, 11, 14, 17, 21 and 24 (including the 4 day induction period). During this time, expression of pluripotency related factor OCT-3/4 was markedly downregulated, whereas expression of PAX-6, ΔNp63α, CK-15 and CK-14 increased prominently, indicating the emergence of limbal epithelial-like cell population (FIG. 2A). Interestingly, ABCG2 was only transiently expressed, peaking at day 9-11 and then gradually decreasing to very low levels by day 24 of differentiation (FIG. 2A). In accordance with the previous results using the protocol (Hongisto et al. 2017), mature corneal epithelial marker CK-12 remained undetectable during this characterization window (data not shown), further verifying the undifferentiated progenitor phenotype of the cells. Quantification of the protein expression from cytospin samples confirmed major differences between the cell populations in the day 10 and day 24 time points (FIG. 2B): For the representative hESC line (Regea08/017), expression of ABCG2 decreased from 62.3% (SD 6.7) to 1.8% (SD 0.9), while the expression of ΔNp63α (as demonstrated by double staining with ΔNp63 and p63α antibodies, FIG. 2C) increased from 23.2% (SD 14.1) to 54.3% (SD 6.2). Expressions of CK15 and CK14 increased from undetectable levels at day 10 to 37.0% (SD 12.4) and 56.2% (14.3) by day 24, respectively. OCT3/4, on the other hand, was expressed in less than 1.5% of the cells already at day 10 and was further diminished to under 1% by day 24. Due to distinct behavior of ABCG2 and ΔNp63α during the differentiation process, we characterized the colocalization of ABCG2 with p63α at day 10 and day 24 cytospin samples. Interestingly, the most intensive staining for both markers was typically observed in different cells, as demonstrated in FIG. 2D. Moreover, at day 10 ABCG2 and p63α colocalized in 31.6% of the cells, whereas at day 24 there were only 1% of the cells that were ABCG2+/p63α +(FIG. 2E). However, the differences observed between the two time points were not statistically significant (Mann-Whitney U test).

To verify the expression pattern of ABCG2, fluorescence activated cell sorting (FACS) analysis was performed from UD-hPSCs as well as day 10-11 and day 24-26 hPSC-LSCs. Indeed, both UD-hPSCs and more differentiated day 24-25 hPSC-LSCs had low expression for ABCG2 (0.8%, SD 1.3 and 1.5%, SD 2.0, respectively, for the representative hESC line Regea08/017), whereas day 10-11 hPSC-LSCs expressed significantly higher levels of ABCG2 (21.6%, SD 8.2, Mann-Whitney U test) (FIG. 2F). Additionally, changes in the ABCG2 expression at transcriptional level were analyzed with qRT-PCR, which also confirmed notably higher expression level of ABCG2 mRNA in day 10 hPSC-LSC population in comparison to UD-hPSCs and day 24 hPSC-LSC population (FIG. 2G). Taken together, the characterization analyses demonstrated very distinct expression profiles for proposed limbal epithelial stem cell/progenitor markers ABCG2, ΔNp63α, CK-15 and CK-14 between day 10-11 and day 24 time points during hPSC-LSC differentiation process.

### EXAMPLE 2

### Maintenance of ABCG2 Expression

These experiments were carried out in order to establish culture conditions required for preserving ABCG2 expression for extended periods.

### Materials and Methods

Culture Conditions for Maintaining ABCG2-Positive hPSC-LSC Phenotype

For the maintenance of ABCG2-positive population, CnT-30 culture medium was replaced with CnT-07 (CELLnTEC Advanced Cell Systems AG, Bern, Switzerland) supplemented with ENRC (50 ng/ml mouse recombinant epidermal growth factor (EGF, Invitrogen), 100 ng/ml mouse recombinant Noggin, 1 μg/ml human recombinant R-spondin-1 (both from Peprotech) and 3 μM CHIR-99021 (Stemgent) at day 10-11 (including the four day induction period). The CnT-07+ENRC medium was either introduced directly to the adherent cultures or alternatively, the hPSC-LSCs were concomitantly enzymatically dissociated to single cell suspension and passaged onto fresh LN-521/Col IV coated wells in a density of 1000-5000 cells/cm² in the new medium. In both cases, the hPSC-LSCs were thereafter cultured following a standard feeding regimen. After emergence of ABCG2-positive colonies, further expansion and/or continued culturing of the hPSC-LSCs in CnT-07+ENRC was achieved by passaging the subconfluent cultures onto fresh LN-521/Col IV coated matrices in a density 1000 cells/cm². Cryopreservation of the cells was carried out as described in Hongisto et al. 2017 Stem Cell Res Ther.

Preservation of the high ABCG2-expression in hPSC-LSC colonies cultured in Cnt-07+ENRC was confirmed in all three studied hPSC lines by IF. Quantitative RT-PCR analysis was carried out for Regea08/017 and UTA.04607.WT. Implementation of the said characterization methods essentially followed those described in Example 1.

In addition to the above, following modified culture conditions for maintaining the ABCG2-positive hPSC-LSC phenotype were tested using hESC line Regea08/017:

In the first related experiment, different cell culture media were tested as basal media with ENRC supplementation. The tested basal media included commercial pluripotent stem cell culture media E8 Flex (Thermo Fisher Scientific), commercial corneal differentiation media CnT-30 (CELLnTEC), and self-formulated xeno-free basal media XF-Ko-SR described in Hongisto et al. 2017 Stem Cell Res & Ther.

In the second related experiment, CnT-07 was used as basal cell culture media supplemented with modified ENRC formulations. In each experimental condition, one component of the ENRC was not provided. Thus, the tested conditions were CnT-07+ENR (without CHIR-99021), CnT-07+ENC (without R-Spondin-1), CnT-07+ERC (without Noggin) and CnT-07+NRC (without EGF).

In both above described experiments, the tested cell culture conditions were introduced directly to the adherent hPSC-LSC cultures at day 11 (including the 4-day differentiation period) and cultured for 10 days, following the standard feeding regimen of three times a week. At the endpoint of the experiments, the cell morphology was assessed and representative images were captured using Nikon Eclipse TE2000-S phase contrast microscope (Nikon Instruments Europe). In the second experiment, the cells were fixed and subjected to IF staining with ABCG2 and p63α antibodies to confirm the maintenance of ABCG2-positive hPSC-LSC phenotype.

### Proliferation Analyses

Population doublings were calculated for Regea11/013 in the end of each subculture using the following formula: log(N/N0)/log2, where N0 is the number of plated cells and N is the number of cells at the end of culture period. Similarly, population doubling time for each passage was calculated with the following formula: T*log2/log(N−N0), where T is the duration of the culture in hours.

### Results

Significantly, replacing the corneal differentiation media CnT-30 with epithelial maintenance media CnT-07, supplemented with specific combination of EGF, Noggin, R-spondin-1 and CHIR-99201 (ENRC), resulted in preservation of colonial morphology and strong ABCG2 expression. Other tested cell culture mediums, namely E8 Flex, CnT-30 and XF-Ko-SR, supplemented with complete ENRC were not as efficient in retaining and expanding the desired cell morphology in relation to unwanted cell types (data not shown). Despite less efficient performance, also these media are suitable for use in the present invention, at least in some embodiments thereof. Interestingly, in addition to CnT-07 supplemented with complete combination of ENRC, ABCG2-positive hPSC-LSC phenotype was also maintained in all tested ENRC-modified conditions that included CHIR-99021, namely CnT-07+ENC, CnT-07+ERC and CnT-07+NRC.

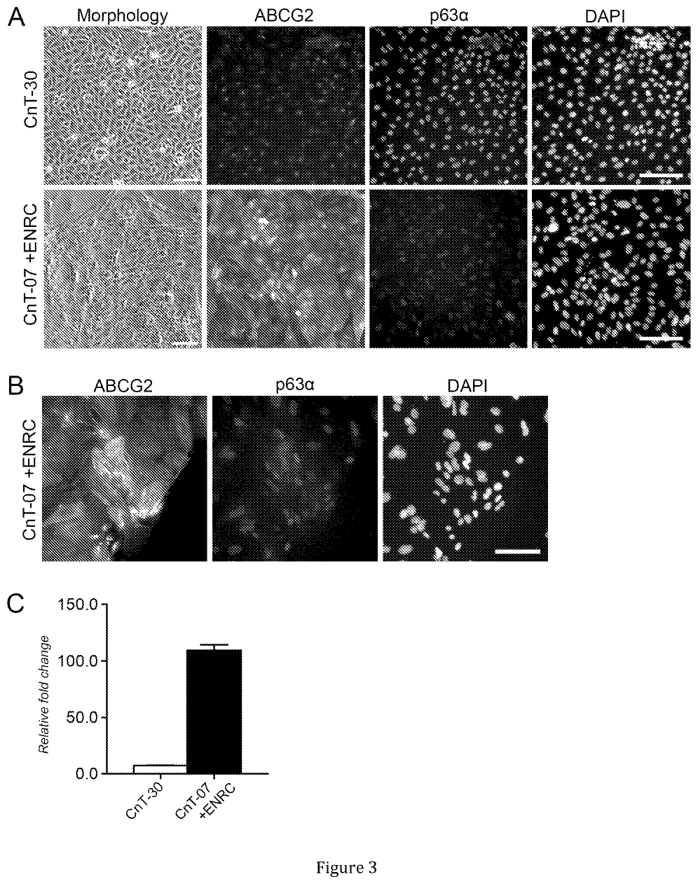

Noteworthy, colonies positive for ABCG2 expressed only low levels of p63α, whereas ABCG2-negative cells in CnT-30 condition were p63α-bright. (FIG. 3A) Furthermore, in CnT-07+ENRC condition ABCG2 expression was maintained even after passaging the cells (FIG. 3B), whereas continued culturing and passaging in CnT-30 differentiation media caused the cells to lose robust ABCG2 expression and differentiate further towards p63α-bright phenotype, as described within the hPSC-LSC differentiation process in Example 1. These IF results were confirmed with three genetically distinct cell lines: two hESC lines (Regea08/017 and Regea11/013) and one hiPSC line (UTA.04607.WT). Effect of the CnT-07+ENRC condition was consistent in all three cases, although differences in the growth efficacy over further passaging as well as in tendency to generate other cell types were observed between the lines. Without passaging, ABCG2-positive colonies persisted at least 24 days in CnT-07+ENRC (35 days of total hPSC-LESC culture time), which was the longest analyzed time point tested with one line, Regea08/017 (data not shown).

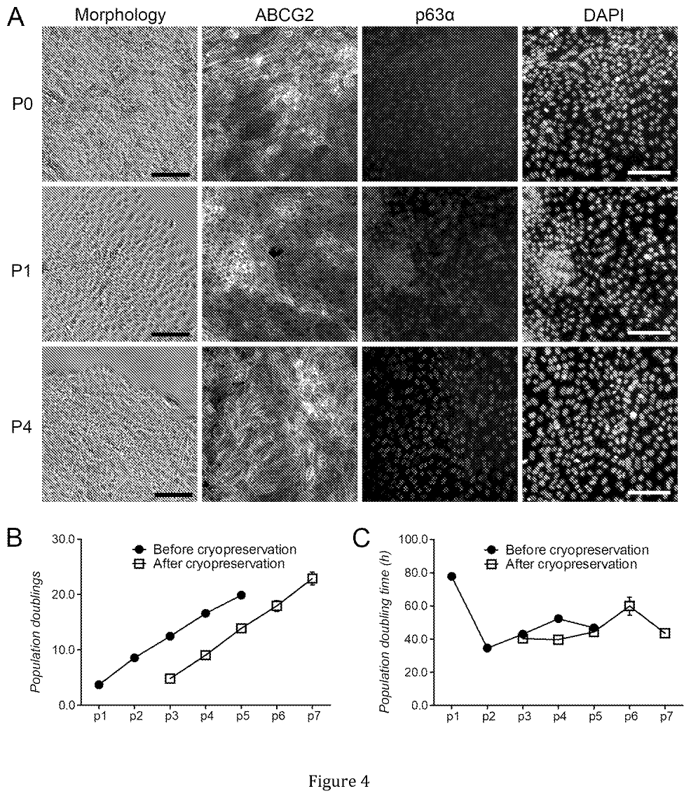

Two different time points were tested in order to determine the optimal time point for passaging the ABCG2-positive hPSC-LSCs. At first, the passaging was carried out at day 21, when the cells had been cultured in CnT-07+ENRC for ten days and expression of ABCG2 had been stabilized. In an alternative approach, the cells were passaged at day 11 (that is, at the peak of the ABCG2 expression window during hPSC-LESC differentiation) and reseeded in CnT-07+ENRC for further culturing. Maintenance of ABCG2-positive colonies was achieved using both methods and was confirmed with all three cell lines, although there were major cell line-specific differences in the growth efficacy. With Regea11/013, the latter approach lead to the formation of highly proliferative colonies and almost no contaminating cells of other cell types. The morphology and ABCG2/p63α expression of the colonies remained unaffected upon further passaging (FIG. 4A). Over twenty population doublings (PD) were observed for Regea11/013 during five passages, average population doubling time (PDT) being 50.9 hours (SD 16.4). (FIG. 4B-C, black dot line). Additionally, cryopreservation between the passages 2 and 3 (p2-p3) did not have a marked effect to the proliferation capacity of the cells, as demonstrated by the white box line in FIGS. 4B-C.

1. A method of maintaining ABCG2-positive phenotype of corneal limbal stem cells, wherein the method comprises culturing ABCG2-positive corneal limbal stem cells in a culture medium comprising EGF and at least one Wnt activator and wherein the ABCG2-positive corneal limbal stem cells do not express cytokeratin 3 or cytokeratin 12.

2. The method according to claim 1, wherein the Wnt activator is selected from the group consisting of GSK3 inhibitors and proteins of R-spondin family.

3. The method according to claim 1, wherein the Wnt activator is CHIR99021.

4. The method according to claim 1, wherein the culture medium further comprises noggin or its supplement LDN-193189.

5. The method according to claim 1, wherein the ABCG2-positive corneal limbal stem cells are ABCG2-positive primary corneal limbal stem cells or pluripotent stem cell-derived ABCG2-positive corneal limbal stem cells.

6. A method of producing ABCG2-positive corneal limbal stem cells, the method comprising:
a) providing pluripotent stem cells;
b) culturing said cells in a cell culture medium comprising a TGF-beta inhibitor and a fibroblast growth factor (FGF);
c) withdrawing the TGF-beta inhibitor and the FGF, and culturing the cells obtained in step b) in a cell culture medium comprising bone morphogenetic protein 4 (BMP-4) thereby producing eye precursor cells;
d) culturing said eye precursor cells in a corneal differentiation medium thereby producing ABCG2-positive corneal limbal stem cells; and
e) culturing said ABCG2-positive corneal limbal stem cells in a maintenance cell culture medium comprising EGF and at least one Wnt activator, thereby maintaining ABCG2-positive corneal limbal stem cell phenotype obtained via steps a-d).
wherein the ABCG2-positive corneal limbal stem cells do not express cytokeratin 3 or cytokeratin 12.

7. The method according to claim 6, wherein the Wnt activator is selected from the group consisting of GSK3 inhibitors and proteins of R-spondin family.

8. The method according to claim 6, wherein the Wnt activator is CHIR99021.

9. The method according to claim 6, wherein the maintenance culture medium in step e) further comprises noggin or its supplement LDN-193189.

10. The method according to claim 6, wherein the pluripotent stem cells are selected from induced pluripotent stem (iPS) cells and embryonic stem (ES) cells, with the proviso that if human embryonic stem (hES) cells are used, the method does not include the destruction of human embryos.

11. The method according to claim 6, wherein step a) comprises forming embryoid bodies from said pluripotent stem cells.

12. The method according to claim 11, wherein said forming of embryoid bodies in step a) is carried out by a physical or chemical method.

13. The method according to claim 6, wherein said culturing in step d) and e) is carried out on a substrate coated at least with collagen IV and laminin.

14. The method according to claim 1, which is performed in substantially xeno-free, substantially serum-free and/or defined conditions.

15. A method for treating an eye disease, comprising administering an effective dose to a patient in need thereof of ABCG2-positive corneal limbal stem cells, whose ABCG2-positive phenotype has been maintained by the method of claim 1.

16. The method of claim 15, wherein the ABCG2-positive corneal limbal stem cells are capable of maintaining their ABCG2-positive phenotype for at least 35 days.

17. The method according to claim 2, wherein the Wnt activator is CHIR99021.

18. The method according to claim 2, wherein the culture medium further comprises noggin or its supplement LDN-193189.

19. The method according to claim 3, wherein the culture medium further comprises noggin or its supplement LDN-193189.

20. The method according to claim 2, wherein the ABCG2-positive corneal limbal stem cells are ABCG2-positive primary corneal limbal stem cells or pluripotent stem cell-derived ABCG2-positive corneal limbal stem cells.
